(19) United States
(12) Patent Application Publication
(10) Pub. No.: US 2008/0237035 A1
(43) Pub. Date: Oct. 2, 2008

(54) Air cleaning apparatus

(75) Inventors: Koichi Kurusu, Gunma (JP); Kohei Nozawa, Gunma (JP); Minco Ikematsu, Ibaraki (JP); Hironobu Sekine, Gunma (JP); Yuko Nowatari, Hyogo (JP); Yoshiaki Noguchi, Gunma (JP); Toru Kawabata, Gunma (JP)

Correspondence Address: MCDERMOTT WILL & EMERY LLP, 600 13TH STREET, N.W., WASHINGTON, DC, 20005-3096, US

(73) Assignee: SANYO ELECTRIC CO., LTD.

(21) Appl. No.: 12/078,247

(22) Filed: Mar. 28, 2008

(30) Foreign Application Priority Data
Mar. 28, 2007 (JP) 2007-84188

(51) Int. Cl. C25B 9/00; A61L 9/00

(52) U.S. Cl. 204/242; 422/120

(57) ABSTRACT

There is disclosed an air cleaning apparatus usable regardless of seasons, weather, environmental conditions and the like. The air cleaning apparatus brings air to be treated into contact with a cleaning solution including active oxygen species to purify the air to be treated includes a water tank which stores the cleaning solution, and a temperature controller which controls a temperature of the cleaning solution stored in the water tank. The temperature controller includes a heat exchanger as a cooling/heating unit which cools or heats the cleaning solution stored in the water tank, and controls the temperature of the cleaning solution into 0° C. or more to 40° C. or less.

### BACKGROUND OF THE INVENTION

1. Field of the Invention

The present invention relates to an air cleaning apparatus which removes a toxic substance, dust and the like included in air to be treated.

2. Description of the Related Art

In recent years, along with popularization of highly dense and highly insulating houses, health disorder such as hypersensitivity to chemical substances is increasing. The chemical substances which cause this hypersensitivity include a substance such as formaldehyde generated from an indoor building material of a wall, a wallpaper or the like, and a substance such as an exhaust gas which flows from the outside. As a apparatus which removes such chemical substances included in air, a water cleaning type air cleaning apparatus has been developed which brings a cleaning solution including active oxygen species such as hypochlorous acid and ozone generated by an electrolytic technology into contact with air to be treated as a target to purify the air.

In the above air cleaning apparatus, as compared with a conventional filter type air cleaning apparatus which traps the chemical substances and the like in the air to be treated with a conventional filter, the active oxygen species can be brought into three-dimensional contact with the air to be treated, so that a large amount of air can be treated at once. Moreover, the apparatus has an excellent characteristic that toxic substances in the air to be treated can be decomposed with the active oxygen species (e.g., see Japanese Patent Application Laid-Open No. 2005-7307).

However, according to such a water cleaning type air cleaning apparatus, when water freezes in winter and at midwinter (especially below freezing point), the active oxygen species cannot be formed by the electrolytic technology or the formed active oxygen species cannot be sprayed, so that there is a problem that the air cannot be cleaned.

On the other hand, when the apparatus is used in summer or in a tropical district, the apparatus is of the water cleaning type, and hence the treated air contains a large amount of water content and becomes highly humid air, so that there is a problem that a user feels uncomfortable. Furthermore, with a rise of a water temperature, a solubility, in water, of the toxic substance included in the air to be treated lowers, so that there is also a problem that a removal efficiency of the toxic substance from the air to be treated lowers.

Therefore, it has been difficult to use the conventional water cleaning type air cleaning apparatus in an environment where the water freezes in winter and at midwinter, or in summer or in the tropical district as described above.

### SUMMARY OF THE INVENTION

The present invention has been developed to solve such a conventional technical problem, and an object thereof is to provide an air cleaning apparatus usable regardless of seasons, weather, environmental conditions and the like.

According to a first aspect of the invention, there is provided an air cleaning apparatus which brings air to be treated into contact with a cleaning solution including active oxygen species to purify the air to be treated, characterized by comprising: a water tank which stores the cleaning solution; and a temperature controller which controls a temperature of the cleaning solution stored in this water tank.

The air cleaning apparatus according to a second aspect of the invention is characterized in that in the above invention, the temperature controller includes cooling/heating means for cooling or heating the cleaning solution stored in the water tank, and controls the temperature of the cleaning solution into 0° C. or more to 40° C. or less.

The air cleaning apparatus according to a third aspect of the invention is characterized in that in the above inventions, the temperature controller controls the temperature of the cleaning solution into 5° C. or more to 15° C. or less.

The air cleaning apparatus according to a fourth aspect of the invention is characterized in that in the second aspect of the invention, the temperature controller controls the temperature of the cleaning solution into 20° C. or more to 25° C. or less.

The air cleaning apparatus according to a fifth aspect of the invention is characterized in that in the first aspect of the invention, the temperature controller includes dehumidifying means for dehumidifying the air to be treated brought into contact with the cleaning solution and then supplied to an air supply space.

The air cleaning apparatus according to a sixth aspect of the invention is characterized in that the above inventions further comprises means for collecting, in the water tank, water condensed and formed by the dehumidifying means.

The air cleaning apparatus according to a seventh aspect of the invention is characterized in that in the above first aspect of the invention, the cleaning solution is obtained by electrolyzing the water in the water tank.

The air cleaning apparatus according to an eighth aspect of the invention is characterized in that the above invention, the water tank includes a depositing section which collects the cleaning solution brought into contact with the air to be treated, and an electrolysis section connected to this depositing section and provided with electrodes which electrolyze the water in the water tank, and the depositing section has a drain port opened/closed by a valve, and tilts downward to this drain port.

The air cleaning apparatus according to a ninth aspect of the invention is characterized in that in the above inventions, each of the active oxygen species is one selected from the group consisting of hypochlorous acid, ozone, hydroxyl radicals and combinations thereof.

According to the first aspect of the invention, the air cleaning apparatus which brings the air to be treated into contact with the cleaning solution including the active oxygen species to purify the air to be treated comprises the water tank which stores the cleaning solution, and the temperature controller which controls the temperature of the cleaning solution stored in this water tank. For example, as in the second aspect of the invention, the temperature controller includes the cooling/heating means for cooling or heating the cleaning solution stored in the water tank, and controls the temperature of the cleaning solution into 0° C. or more to 40° C. or less. In this case, regardless of seasons, weather, environmental conditions and the like, the air cleaning apparatus can be used in any region of the world throughout the year.

In particular, in a case where the air cleaning apparatus is used in an environment in which air temperature is below freezing point in winter or at midwinter, as in the third aspect of the invention, the temperature controller controls the temperature of the cleaning solution into 5° C. or more to 15° C. or less, whereby a disadvantage that the cleaning solution freezes can be avoided, and the air cleaning apparatus can efficiently be operated.

Furthermore, in a case where the air cleaning apparatus is used in an environment in which the air temperature is high, for example, in summer or in a tropical district, as in the fourth aspect of the invention, the temperature controller controls the temperature of the cleaning solution into 20° C. or more to 25° C. or less, whereby a disadvantage that a removal efficiency of a toxic substance lowers with rise of a water temperature owing to lowering of solubility in water can be avoided, and the air cleaning apparatus can efficiently be operated.

Moreover, as in fifth aspect of the invention, the temperature controller includes the dehumidifying means for dehumidifying the air to be treated brought into contact with the cleaning solution and then supplied to the air supply space, whereby the dehumidified air to be treated can be supplied to the air supply space, and comfort can be improved.

In particular, as in the sixth aspect of the invention, the air cleaning apparatus further comprises the means for collecting, in the water tank, the water condensed and formed by the dehumidifying means, whereby the water to be supplied to the water tank can be saved.

Moreover, as in the seventh aspect of the invention, the cleaning solution is obtained by electrolyzing the water in the water tank. In consequence, it is possible to solve a problem of procurement cost of a commercially available aqueous active oxygen species as in a case where the species are used as the cleaning solution. It is also possible to solve problems of danger during handling and storage as in a case where the aqueous active oxygen species are prepared using a reagent.

Furthermore, as in the eighth aspect of the invention, the water tank includes the depositing section which collects the cleaning solution brought into contact with the air to be treated, and the electrolysis section connected to this depositing section and provided with the electrodes which electrolyze the water in the water tank, and the depositing section has the drain port opened/closed by the valve, and tilts downward to this drain port. In consequence, sediments such as soil and sandblast collected from the air to be treated and deposited in the water tank can be discharged from the drain port.

Moreover, in the above inventions, as in the ninth aspect of the invention, each of the active oxygen species is one selected from the group consisting of hypochlorous acid, ozone, hydroxyl radicals and combinations thereof. In consequence, the toxic substance can efficiently be decomposed with the cleaning solution including the active oxygen species to remove the toxic substance.

### BRIEF DESCRIPTION OF THE DRAWINGS

FIG. 1 is a constitution diagram of an air cleaning apparatus according to one embodiment of the present invention (Embodiment 1);

FIG. 2 is a schematic diagram showing an operation of the air cleaning apparatus of FIG. 1 in summer;

FIG. 3 is a flow chart showing control of a supply pump of a circulation path shown in FIG. 2;

FIG. 4 is a flow chart showing control of an electromagnetic valve of bypass piping shown in FIG. 2;

FIG. 5 is a flow chart showing control of a freezing unit shown in FIG. 2;

FIG. 6 is a schematic diagram showing an operation of the air cleaning apparatus of FIG. 1 in winter;

FIG. 7 is a flow chart showing control of a supply pump of a circulation path shown in FIG. 6;

FIG. 8 is a flow chart showing control of an electromagnetic valve of a bypass piping shown in FIG. 6;

FIG. 9 is a flow chart showing control of a freezing unit shown in FIG. 6;

FIG. 10 is a flow chart showing control of a water tank of the air cleaning apparatus shown in FIG. 1;

FIG. 11 is a diagram showing a correlation between a temperature and reactivity of ozone and hypochlorous acid against a toxic substance;

FIG. 12 is a diagram showing a correlation between a temperature and solubility of a toxic substance (an ammonia solution) in water; and

FIG. 13 is a diagram showing a result of removal of the toxic substance by use of the air cleaning apparatus.

### DESCRIPTION OF THE PREFERRED EMBODIMENT

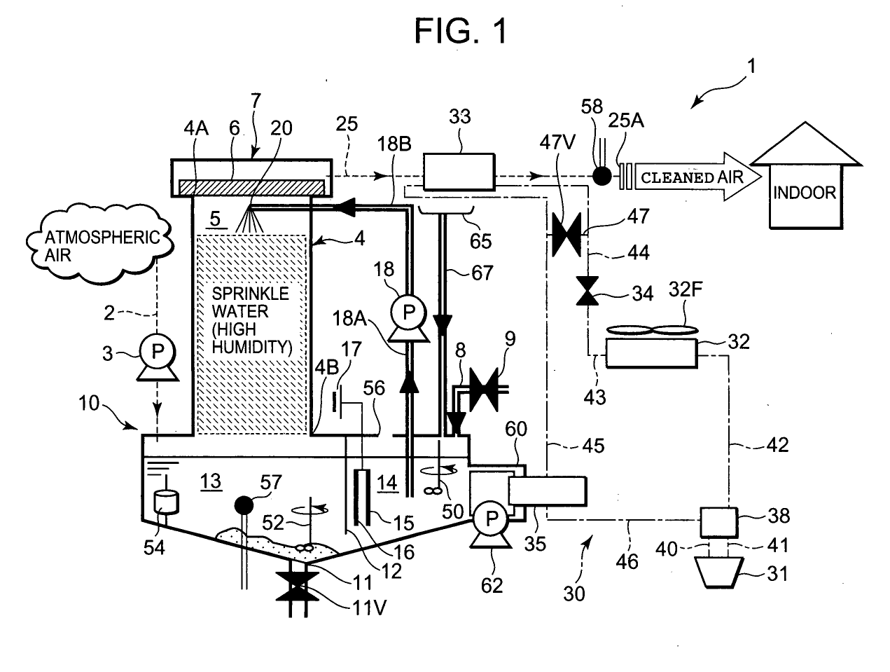

An embodiment of the present invention will hereinafter be described with reference to the drawings. FIG. 1 is a constitution diagram of an air cleaning apparatus 1 according to one embodiment of the present invention. The air cleaning apparatus 1 of the embodiment is installed in an outside air introduction path which takes outside air into a highly airtight house or the like, and the apparatus brings air to be treated into contact with a cleaning solution to purify the air to be treated introduced indoors. The air includes toxic substances such as odor, pollen, allergen, a VOC, a pesticide and oxidant, fine matters such as soil and sandblast and the like. The apparatus is constituted of a gas-liquid contact chamber 5 for trapping the toxic substances included in the air to be treated, a water tank 10 which stores a cleaning solution and the like.

The gas-liquid contact chamber 5 is constituted in a cleaning tower 4 including a cylinder, a square column or the like, an upper end of the chamber is provided with an exhaust port 4A, and a lower end thereof is provided with a suction port 4B. A shower head 20 for jetting the cleaning solution toward a lower part of the gas-liquid contact chamber 5 so that the solution drops down is disposed in an upper part of this gas-liquid contact chamber 5 (an inner part of the gas-liquid contact chamber 5 in the vicinity of the exhaust port 4A). A lid member 7 is attached to an upper part of the cleaning tower 4, and is provided with a draft mesh 6 for splash prevention which covers the exhaust port 4A formed in the upper end of the gas-liquid contact chamber 5. This mesh 6 for splash prevention is a draft water droplet trap for splash prevention for avoiding a disadvantage that water sprinkled from the shower head 20 flies upward to the upper part of the cleaning tower 4, and is discharged from the exhaust port 4A together with the air to be treated. The mesh is constituted of a metal which does not easily deteriorate or erode owing to the cleaning solution, a mesh-like material made of a resin or the like, a plate member provided with a plurality of holes or the like.

Moreover, an opening is formed in a side surface of the lid member 7 above this mesh 6 for splash prevention, and the opening is connected to one end of an air supply duct 25. It is constituted that the air to be treated passed through the mesh 6 for splash prevention can enter the air supply duct 25 via the lid member 7. The other end of this air supply duct 25 is an outlet (hereinafter referred to as a discharge port) 25A of the air to be treated, and opens in a room (indoors) as an air supply space.

On the other hand, the water tank 10 is installed under the cleaning tower 4. This water tank 10 stores the cleaning solution which has dropped down from the shower head 20 so that the solution is circulated again through the shower head 20, and the water tank is connected to the suction port 4B in the lower end of the cleaning tower 4.

An inner upper part of the water tank 10 is divided into two sections by a partition wall 12, one section (on the left side in FIG. 1) is a depositing section 13, and the other section (on the right side in FIG. 1) is an electrolysis section 14. The depositing section 13 is provided right under the gas-liquid contact chamber 5 of the cleaning tower 4, and is constituted so that the cleaning solution brought into contact with the air to be treated in the gas-liquid contact chamber 5 can be collected. This depositing section 13 has a drain port 11 for discharging sediments from the water tank 10, and the whole bottom part of the water tank 10 tilts downward to this drain port 11 so that the sediments are easily discharged from the drain port 11. This drain port 11 is provided with an electromagnetic valve 11V, and the drain port 11 is openably closed by this electromagnetic valve 11V.

Then, the electrolysis section 14 of the water tank 10 is provided with a pair of electrodes 15, 16 (electrolytic units). The electrodes 15, 16 electrochemically treat (electrolyze) tap water stored in the water tank 10, or water to which sodium chloride has been added (i.e., water including chloride ions) to form electrolytic water (a cleaning solution). Specifically, the electrodes 15, 16 electrolyze the water (the tap water in the present embodiment) in the water tank 10 owing to power supply from a power source 17 to form the electrolytic water (the cleaning solution) including active oxygen species. That is, when a predetermined voltage is supplied from the power source 17 to the electrodes 15, 16, the tap water in the water tank 10 is electrolyzed to form the electrolytic water (the cleaning solution) including the active oxygen species.

In the present embodiment, diamond electrodes are used as the electrodes 15, 16. The tap water is electrolyzed using such diamond electrodes, whereby the electrolytic water (the cleaning solution) including the active oxygen species can be obtained in the water tank 10.

Here, the active oxygen species include oxygen molecules having oxidation activity higher than usual oxygen, and related substances. Active oxygen in a so-called narrow sense such as super oxide anions, singlet oxygen, hydroxyl radicals or hydrogen peroxide includes active oxygen in a so-called broad sense such as, for example, ozone or hypohalogenous acid. It is to be noted that the active oxygen species formed in the present embodiment include one of the group consisting of the hypochlorous acid, ozone, hydroxyl radials and combinations thereof.

It is to be noted that in the present embodiment, the tap water in the water tank 10 is electrolytically treated, whereby the electrolytic water including the active oxygen species is formed, and is used as the cleaning solution. However, an aqueous active oxygen species solution such as a commercially available hypochlorous acid solution or ozone water may be supplied to the water tank 10 for use as the cleaning solution. However, in a case where the commercially available aqueous active oxygen species solution is used as the cleaning solution, there are problems that much procurement cost of the aqueous active oxygen species solution is required and that the solution is not easily available. When the aqueous active oxygen species solution is prepared using a reagent, there are problems of danger during handling of the reagent, storage and the like. Furthermore, in a case where gas-phase ozone is formed from air by plasma discharge or the like, and is dissolved in water to form the ozone water, there is a problem that a concentration of the ozone water cannot be set to a sufficiently high concentration.

In consideration of these respects, it is most preferable to form the electrolytic water including the active oxygen species by the electrolytic treatment as in the present embodiment. The electrodes for use are not limited to the diamond electrodes of the present embodiment, and metal electrodes made of platinum, iridium or the like or coated with platinum, iridium or the like may be used.

On the other hand, reference numeral 18 is a supply pump for pumping up the electrolytic water (the cleaning solution) formed by the electrolysis section 14 of the water tank 10 and then allowing the water to drop down from the shower head 20. A suction side of the supply pump 18 is connected to a suction pipe 18A, and a lower end of this suction pipe 18A opens in the electrolytic water (the cleaning solution) of the electrolysis section 14 of the water tank 10. A discharge side of the supply pump 18 is connected to a supply pipe 18B, and an upper end of this supply pipe 18B is connected to the shower head 20. Then, the supply pump 18 pumps up the electrolytic water from the electrolysis section 14 of the water tank 10, and this electrolytic water is sprinkled from the shower head 20 into the gas-liquid contact chamber 5.

Moreover, one end of the water tank 10 on the side of the depositing section 13 is connected to an outside air introduction passage 2 for introducing atmospheric air (outside air) into the air cleaning apparatus 1. In this outside air introduction passage 2, there is installed a blower 3 for sucking air (the atmospheric air) from the outside of the air cleaning apparatus 1 to supply the air to the water tank 10. One end of the outside air introduction passage 2 is connected to an upper part of the depositing section 13 of the water tank 10, and opens above water surface in the water tank 10. The other end of the outside air introduction passage 2 opens in the outside of the air cleaning apparatus 1. Then, when the blower 3 is operated, the air (the atmospheric air) is sucked from the other end of the outside air introduction passage 2, and this sucked air is discharged on the water surface in the water tank 10.

Furthermore, the other end of the water tank 10 on the side of the electrolysis section 14 is connected to a water supply passage 8 for supplying water into the water tank 10. One end of this water supply passage 8 is connected to an upper part of the electrolysis section 14 of the water tank 10, and opens above the water surface in the water tank 10. The water supply passage 8 exits from the water tank 10 from one end thereof which opens in this water tank 10, and the other end of the water supply passage is connected to a water source of the tap water or the like via a water supply valve 9 (an electromagnetic valve). Then, the water supply valve 9 is opened or closed, whereby the tap water can be supplied from the water source into the water tank 10.

It is to be noted that in FIG. 1, 50 is a stirring rod for stirring water (hereinafter referred to as the cleaning solution) in the water tank 10, 52 is a deposit stirring rod for stirring sediments deposited in the water tank 10, 54 is a water level sensor for detecting a water level of the cleaning solution in the water tank 10, and 56 is an air hole for gas venting in the water tank 10.

In addition, in such a water cleaning type air cleaning apparatus, in a case where water freezes in winter or at midwinter (especially below freezing point), the active oxygen species cannot be formed by an electrolytic technology or the formed active oxygen species cannot be sprayed, so that there is a problem that the air cannot be cleaned. On the other hand, when the apparatus is used in summer or in a tropical district, the apparatus is of the water cleaning type, and hence the treated air contains a large amount of water content and becomes highly humid air, so that there is a problem that a user feels uncomfortable.

Furthermore, with regard to the toxic substance in the air to be treated, a solubility in water lowers with a rise of a water temperature, so that there is a problem that a removal efficiency of the toxic substance from the air to be treated lowers. Thus, the conventional water cleaning type air cleaning apparatus has a large problem, when used in water, at midwinter, in summer or in the tropical district.

To solve the problem, the air cleaning apparatus 1 of the present invention includes a temperature controller which controls a temperature of the cleaning solution stored in the water tank 10 so that the above-mentioned problem can be solved and so that the air can preferably be cleaned in any environment or district. The temperature controller of the present embodiment includes a third heat exchanger 35 of a freezing cycle 30 as cooling/heating means for cooling or heating the cleaning solution in the water tank 10, a temperature sensor 57 which detects a temperature of the cleaning solution in the water tank 10, and a temperature and humidity sensor 58 for detecting a temperature and a humidity of the air to be treated supplied indoors.

The temperature sensor 57 is installed in the water tank 10, and the temperature and humidity sensor 58 is disposed in the vicinity of a discharge port 25A of the air supply duct 25.

Moreover, the freezing cycle 30 includes a compressor 31, a four-way valve 38, a first heat exchanger 32, an expansion valve 34 as a pressure reduction unit, a second heat exchanger 33, the third heat exchanger 35 and the like, and these components are successively connected in an annular form via pipes to constitute a well-known refrigerant circuit. That is, a refrigerant discharge pipe 41 connected to a discharge side of the compressor 31 is connected to the four-way valve 38. This four-way valve 38 is channel control means which performs control so that a refrigerant compressed by the compressor 31 is circulated through the first heat exchanger 32 to suck the refrigerant from the third heat exchanger 35 into the compressor 31 or so that the refrigerant compressed by the compressor 31 is circulated through the third heat exchanger 35 to suck the refrigerant from the first heat exchanger into the compressor 31. This four-way valve 38 is connected to the refrigerant discharge pipe 41, a refrigerant pipe 42, a refrigerant pipe 46 and a refrigerant introduction pipe 40.

The refrigerant pipe 42 connected to this four-way valve 38 is connected to one end of the first heat exchanger 32. This first heat exchanger 32 is an air cooling type heat exchanger including a fan 32F, and is constituted so that heat exchange between the heat exchanger and surrounding air blown by the fan 32F can be performed. A refrigerant pipe 43 connected to the other end of the first heat exchanger 32 reaches one end of the expansion valve 34, and a refrigerant pipe 44 connected to the other end of the expansion valve 34 is connected to one end of the second heat exchanger 33.

This second heat exchanger 33 is installed in the air supply duct 25 so that heat exchange between the exchanger and the air to be treated flowing through the air supply duct 25 can be performed. The second heat exchanger functions as dehumidification means for dehumidifying the air to be treated brought into contact with the cleaning solution in the water tank 10 and supplied to the air supply space (indoors) via the air supply duct 25. That is, during an operation in summer described later or during a humidifying operation, when the refrigerant having a pressure thereof reduced by the expansion valve 34 flows into the second heat exchanger 33, heat exchanger between the refrigerant and the air to be treated is performed in the second heat exchanger 33, and the air to be treated is cooled. At this time, a water content included in the air is condensed on the surface of the second heat exchanger 33. In consequence, the water content can be removed from the air to be treated.

Moreover, a drain pan 65 for receiving the water content (drainage) condensed and formed by the second heat exchanger 33 is provided under the second heat exchanger 33, and a bottom part of this drain pan 65 is connected to a drain pipe 67, so that the drainage on the drain pan 65 can be collected in the water tank 10 via this drain pipe 67.

On the other hand, a refrigerant pipe 45 connected to the other end of the second heat exchanger 33 is connected to one end of the third heat exchanger 35. This third heat exchanger 35 is a water cooling type heat exchanger provided so as to perform heat exchange between the exchanger and the cleaning solution in the water tank 10. In the present embodiment, the third heat exchanger 35 is installed in a circulation passage 60 formed in an end of the water tank 10 on the side of the electrolysis section 14, and the cleaning solution is supplied from the water tank 10 to the third heat exchanger 35 by a supply pump 62 interposed in this circulation passage 60, so that heat exchange between the refrigerant flowing through the third heat exchanger 35 and the cleaning solution in the water tank 10 can be performed. The other end of the third heat exchanger 35 is connected to the refrigerant pipe 46 connected to the four-way valve 38.

Then, a middle part of the refrigerant pipe 44 which connects the expansion valve 34 to the second heat exchanger 33 is connected to one end of a bypass pipe 47 which bypasses the second heat exchanger 33, and this pipe 47 is provided with an electromagnetic valve 47V for opening and closing the pipe 47. The other end of this pipe 47 is connected to a middle part of the refrigerant pipe 45.

An operation of the air cleaning apparatus 1 according to the present invention having the above constitution will be described. When power supply of the air cleaning apparatus 1 is turned on, energization of the electrodes 15, 16 is started. In consequence, the tap water stored in the water tank 10 is electrolyzed to form the above electrolytic water (the cleaning solution) including the active oxygen species (an electrochemical treatment).

Then, simultaneously with the energization of the electrodes 15, 16, the supply pump 18 and the blower 3 are started. In consequence, the electrolytic water (the cleaning solution) in the water tank 10 is pumped up from the suction pipe 18A by the supply pump 18. This pumped cleaning solution is supplied from the supply pipe 18B to the shower head 20, jetted and sprayed downwards in the blower 3. Subsequently, with the start of the blower 3, the outside air (the air to be treated) is sucked into the outside air introduction passage 2, and discharged toward the water surface in the water tank 10. The air to be treated discharged toward the water surface of this water tank 10 collides with the surface of the cleaning solution, then moves upward in the gas-liquid contact chamber 5 owing to a blow pressure of the blower 3, and passes through the gas-liquid contact chamber 5 in which the cleaning solution has sprayed from the shower head 20.

At this time, toxic substances such as odor, pollen, allergen, a VOC, a pesticide and oxidant included in the air to be treated are brought into contact with the cleaning solution to trap the toxic substance. The substances reach the water tank 10, and are decomposed by the active oxygen species generated by electrolysis in the electrolysis section 14 of the water tank 10. Moreover, the cleaning solution jetted from the shower head 20 into the gas-liquid contact chamber 5 also includes the active oxygen species, so that a part of the toxic substances in the air to be treated comes in contact with the active oxygen species included in the cleaning solution, and is decomposed in the gas-liquid contact chamber 5. Furthermore, fine matters such as soil and sandblast included in the air to be treated passed through the sprayed solution are dissolve in the cleaning solution, and separated from the air to be treated. The separated fine matters reach the water tank 10, are precipitated in the water tank 10, and deposited as sediments.

Then, the air to be treated from which the toxic substances and the fine matters have been removed in the gas-liquid contact chamber 5 as described above passes through the mesh 6 for splash prevention provided above the gas-liquid contact chamber 5. The air to be treated from which a surplus water content has been removed by this mesh 6 for splash prevention is discharged from one end opening of the lid member 7 to the air supply duct 25, and is supplied indoors from the discharge port 25A formed in the other end of the air supply duct 25.

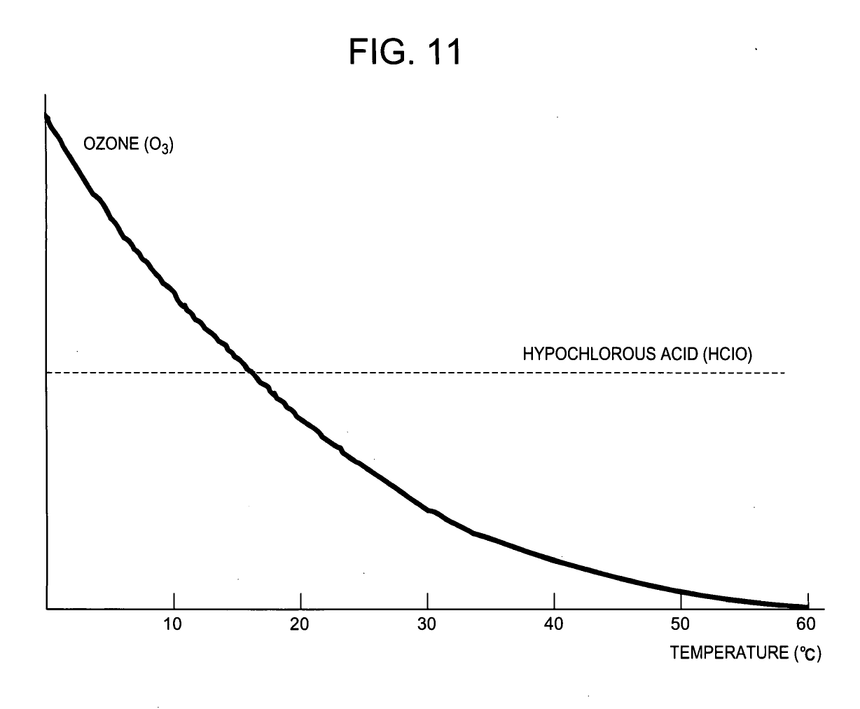

In addition, in the air cleaning apparatus 1 of the present embodiment, the temperature controller controls the temperature of the cleaning solution in the water tank 10 into a predetermined temperature range as described above. In this case, a lower limit temperature of the temperature range is preferably set to a temperature at which the cleaning solution in the water tank 10 does not freeze, that is, 0° C. or more. An upper limit temperature needs to be set to a temperature at which a decomposition efficiency of the toxic substances included in the air to be treated does not remarkably lower. To solve the problem, the temperature of the cleaning solution is changed to verify reactivity to the toxic substances. A result is shown in FIG. 11. In this case, the reactivity of the cleaning solution including ozone and hypochlorous acid with respect to the toxic substances was verified. In FIG. 11, a broken line shows the reactivity of hypochlorous acid with respect to the toxic substances. It has been found that hypochlorous acid has a substantially constant reactivity regardless of the temperature change. In FIG. 11, a solid line shows the solubility of ozone in water. That is, ozone instantly reacts with the toxic substances as targets, so that the reactivity of ozone is substantially equal to the solubility in water. It has been found from the result of FIG. 11 that the solubility in water (i.e., the reactivity to the toxic substances) is high in a case where a temperature of ozone is low and that the reactivity rapidly rises in a case where the temperature lowers to +30° C. or less.

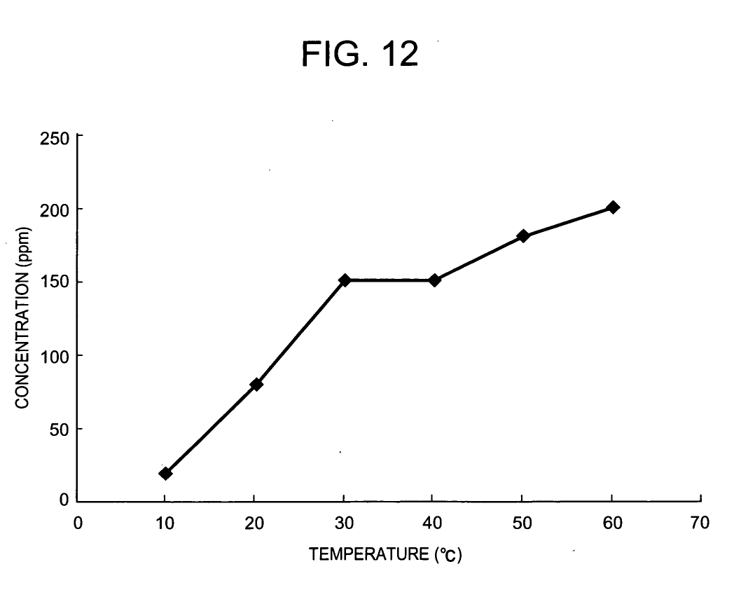

Subsequently, the solubility of the toxic substances in water with the temperature change was verified. FIG. 12 shows a result of measurement of a concentration of ammonia in a gas of a sealed container in a case where an ammonia solution having a concentration of 100 ppm was heated in the container. It has been found in FIG. 12 that the concentration of ammonia in the gas increases in a case where the temperature rises and that the concentration of ammonia in the gas remarkably rises especially in a case where the temperature exceeds 40° C.

It has been found from the above results that the temperature of the cleaning solution in the water tank 10 is controlled into 0° C. or more to 40° C. or less to form the cleaning solution including the active oxygen species, and the solution is brought into contact with the toxic substances, whereby the toxic substances can efficiently be decomposed with the cleaning solution, and can be removed. Therefore, in the present invention, the operation of the freezing unit 30 and the supply pump 62 of the circulation passage 60 are controlled based on outputs of the temperature of the cleaning solution in the water tank 10 detected by the temperature sensor 57 and the temperature and humidity of the air to be treated in the air supply duct 25 detected by the temperature and humidity sensor 58, to control the temperature of the cleaning solution in the water tank 10 into 0° C. or more to +40° C. or less.

Specifically, it is assumed in the present embodiment that the temperature of the cleaning solution is controlled into +20° C. or more to +25° C. or less in summer and that the temperature of the cleaning solution is controlled into +5° C. or more to +15° C. or less in winter. A control operation in this case will be described. Thus, the temperature of the cleaning solution is controlled into +20° C. or more to +25° C. or less in summer, and the temperature of the cleaning solution is controlled into +5° C. or more to +15° C. or less in winter, whereby a power consumption of the freezing unit 30 can be suppressed to perform an efficient operation. First, there will be described an operation of the freezing unit 30 in a case where the air cleaning apparatus 1 of the present embodiment is used in summer when an outside air temperature is in a range of +30° C. to +40° C. (or in a district such as the tropical district).

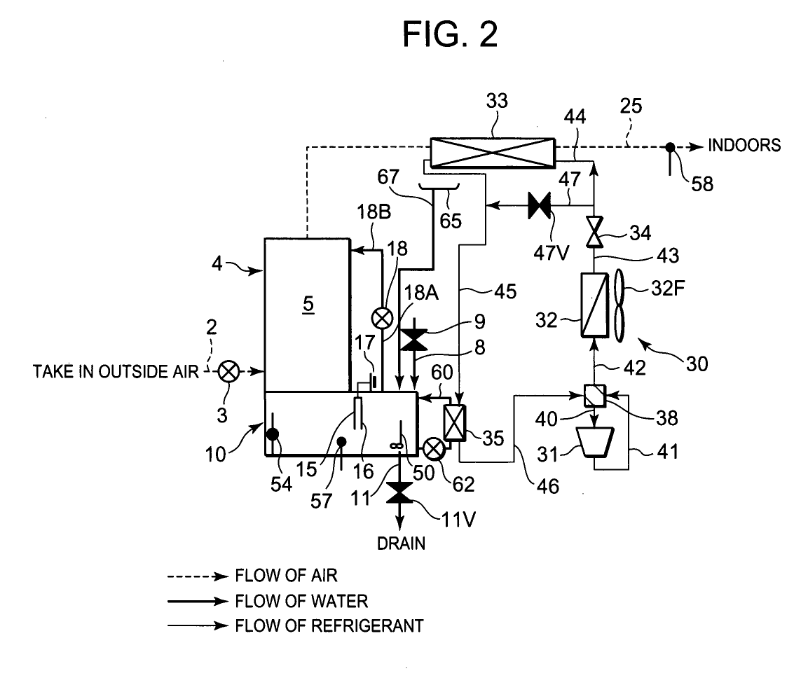

In this case, as shown in FIG. 2, the four-way valve 38 is controlled so that the refrigerant compressed by the compressor 31 flows into the first heat exchanger 32 and so that the refrigerant from the third heat exchanger 35 is sucked into the compressor 31. In consequence, the first heat exchanger 32 functions as a radiator, and the second heat exchanger 33 and/or the third heat exchanger 35 function as an evaporator. It is to be noted that in FIG. 2, broken-line arrows show a flow of the air to be treated, solid-line arrows show a flow of the refrigerant flowing through the freezing unit 30 during the operation in summer, and bold-line arrows show a flow of water, respectively.

That is, the refrigerant compressed by the operation of the compressor 31 and having a high temperature and high pressure is discharged from the compressor 31 via the refrigerant discharge pipe 41, flows into the first heat exchanger 32 through the four-way valve 38, and radiates heat there. Afterward, the pressure of the refrigerant is reduced by the expansion valve 34. When the electromagnetic valve 47V of the bypass pipe 47 is closed, the refrigerant having the pressure thereof reduced by the expansion valve 34 reaches the second heat exchanger 33 installed in the air supply duct 25. Then, the refrigerant absorbs heat from the air to be treated, flowing through the air supply duct 25, in the second heat exchanger 33, and then flows into the third heat exchanger 35.

On the other hand, when the electromagnetic valve 47V is opened and the bypass pipe 47 is opened, the refrigerant having the pressure thereof reduced by the expansion valve 34 does not flow through the second heat exchanger 33, and flows into the third heat exchanger 35 through the bypass pipe 47.

When the supply pump 62 is operated in the third heat exchanger 35 and the cleaning solution in the water tank 10 is supplied to the third heat exchanger 35, the refrigerant absorbs the heat from the cleaning solution supplied by the supply pump 62 in the third heat exchanger 35. In consequence, the cleaning solution is cooled. On the other hand, when the supply pump 62 is stopped, heat exchange between the refrigerant and the cleaning solution is hardly performed, and the refrigerant passes through the third heat exchanger 35, and is sucked into the compressor 31 via the refrigerant introduction pipe 40 through the refrigerant pipe 46 and the four-way valve 38. This cycle is repeated.

Here, during the operation in summer, the operation of the supply pump 62 is controlled, and the electromagnetic valve 47V of the bypass pipe 47 is opened and closed to control the temperature into +20° C. or more to +25° C. or less. That is, the operation of the supply pump 62 is controlled based on the temperature of the cleaning solution in the water tank 10 detected by the temperature sensor 57, and the opening/closing of the electromagnetic valve 47V of the bypass pipe 47 is controlled based on the temperature and humidity of the air to be treated detected by the temperature and humidity sensor 58. A control operation in summer will hereinafter specifically be described in detail.

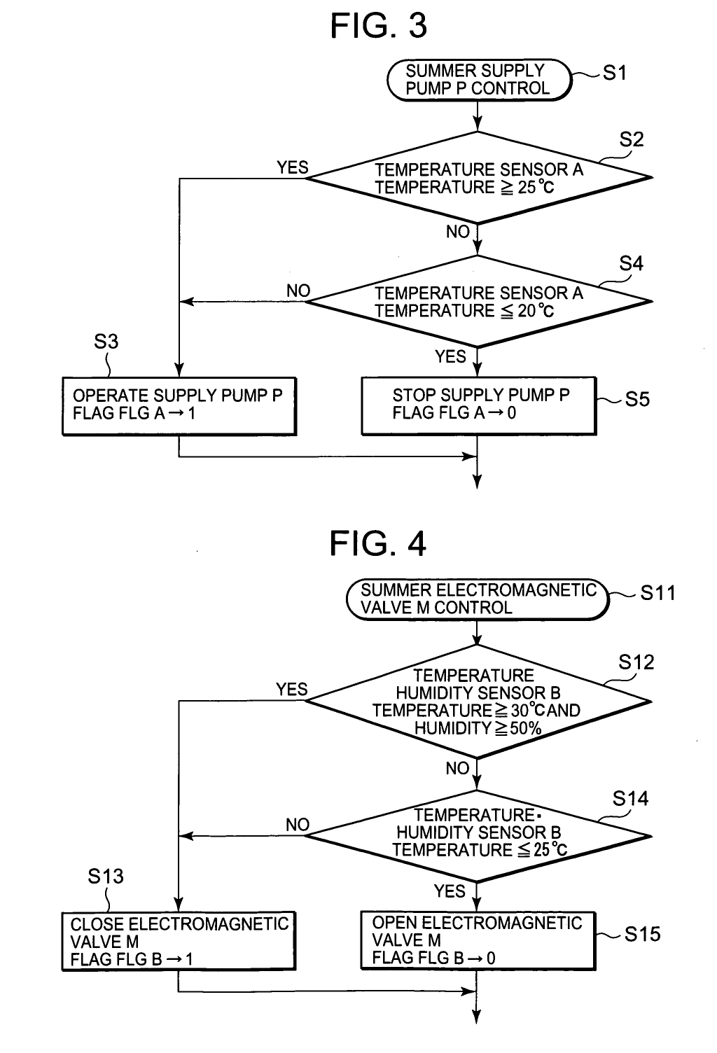

First, the control of the supply pump 62 will be described with reference to FIG. 3. When the power supply of the air cleaning apparatus 1 is turned on and the control of the supply pump 62 (a supply pump P shown in FIG. 3) is started in step S1 of FIG. 3, it is judged in step S2 of FIG. 3 whether or not the temperature of the cleaning solution in the water tank 10 detected by the temperature sensor 57 (a temperature sensor A shown in FIG. 3) is +25° C. or more. Then, when the temperature of the cleaning solution detected by the temperature sensor 57 is +25° C. or more, the supply pump 62 is operated, and a flag FLGA (hereinafter referred to as a flag A) is set to 1 in step S3 of FIG. 3.

Thus, when the temperature of the cleaning solution in the water tank 10 detected by the temperature sensor 57 is +25° C. or more, the supply pump 62 is operated. In consequence, the cleaning solution in the water tank 10 is supplied to the third heat exchanger 35, and heat exchange between the refrigerant and the cleaning solution is performed in the third heat exchanger 35. In consequence, the refrigerant flowing through the third heat exchanger 35 takes heat from the cleaning solution to cool the cleaning solution.

On the other hand, in a case where it is judged in the step S2 of FIG. 3 that the temperature of the cleaning solution detected by the temperature sensor 57 is less than +25° C., it is judged in step S4 of FIG. 3 whether or not an output of the temperature sensor 57 is +20° C. or less. Here, when the output of the temperature sensor 57 is higher than +20° C., in the step S3 of FIG. 3, the supply pump 62 is operated, and the flag A is set to 1 as described above. On the other hand, in a case where it is judged in the step S4 of FIG. 3 that the temperature is +20° C. or less, in step S5 of FIG. 3, the operation of the supply pump 62 is stopped, and the flag A is set to 0, that is, the flag A is reset.

Thus, in a case where the temperature of the cleaning solution in the water tank 10 detected by the temperature sensor 57 lowers to +20° C. or less, the supply pump 62 is stopped, so that the heat exchange between the refrigerant and the cleaning solution is not performed in the third heat exchanger 35.

Next, control of the electromagnetic valve 47V in summer will be described with reference to FIG. 4. When the power supply of the air cleaning apparatus 1 is turned on and the control of the electromagnetic valve 47V (an electromagnetic valve M shown in FIG. 4) is started in step S11 of FIG. 4, it is judged in step S12 of FIG. 4 whether or not the temperature of the air to be treated in the air supply duct 25 detected by the temperature and humidity sensor 58 (a temperature and humidity sensor B shown in FIG. 4) is +30° C. or more, and it is also judged whether or not the humidity of the air to be treated is 50% or more. At this time, in a case where at least one of conditions that the temperature of the air to be treated detected by the temperature and humidity sensor 58 is +30° C. or more and that the humidity is 50% or more is satisfied, in step S13 of FIG. 4, the electromagnetic valve 47V is closed, and a flag FLGB (hereinafter referred to as the flag B) is set to 1.

Thus, in a case where at least one of the conditions that the temperature of the air to be treated detected by the temperature and humidity sensor 58 is +30° C. or more and that the humidity is 50% or more is satisfied, the bypass pipe 47 is closed by the electromagnetic valve 47V. In consequence, the refrigerant having the pressure thereof reduced by the expansion valve 34 does not flow through the bypass pipe 47, and all of the refrigerant flows into the second heat exchanger 33 installed in the air supply duct 25 to absorb heat from the air to be treated flowing around the second heat exchanger 33.

In consequence, the heat is taken from the air to be treated by the refrigerant flowing through the second heat exchanger 33 to cool the air. At this time, the water content included in the air to be treated is condensed on the surface of the second heat exchanger 33. Thus, in a case where at least one of the conditions that the temperature of the air to be treated detected by the temperature and humidity sensor 58 is +30° C. or more and that the humidity is 50% or more is satisfied, the bypass pipe 47 is closed by the electromagnetic valve 47V, and the refrigerant having the pressure thereof reduced by the expansion valve 34 flows into the second heat exchanger 33 installed in the air supply duct 25. Then, the refrigerant which has flowed into the second heat exchanger 33 performs the heat exchange between the refrigerant and the air to be treated flowing around the second heat exchanger, so that this air to be treated can be cooled and dehumidified. Therefore, the air to be treated supplied indoors from the air supply duct 25 can be dehumidified, so that indoor comfort can be improved. In particular, the air to be treated is cooled by the second heat exchanger 33 in summer, so that indoor cooling can be performed or assisted.

Then, the water from the air to be treated condensed and formed on the surface of the second heat exchanger 33 as described above is received as water droplets in the drain pan 65, and collected in the water tank 10 via the drain pipe 67 connected to the bottom part of this drain pan 65. Thus, the drain pan 65 and the drain pipe 67 which connects this drain pan 65 to the water tank 10 are provided, whereby the water condensed and formed by the second heat exchanger 33 can be collected in the water tank 10. In consequence, the water supply to the water tank 10 can be saved.

On the other hand, in a case where it is judged in the step S12 of FIG. 4 that the temperature of the air to be treated in the air supply duct 25 detected by the temperature and humidity sensor 58 is lower than +30° C. and that the humidity of the air to be treated is lower than 50%, it is judged in step S14 of FIG. 4 whether or not the output of the temperature and humidity sensor 58 is +25° C. or less. Then, when the output of the temperature and humidity sensor 58 is higher than +25° C., in the step S13 of FIG. 4, the electromagnetic valve 47V is closed and the flag B is set to 1 as described above.

On the other hand, in a case where it is judged in the step S14 of FIG. 4 that the output of the temperature and humidity sensor 58 is +25° C. or less, in step S15 of FIG. 4, the electromagnetic valve 47V is opened, and the flag B is set to 0 (i.e., the flag B is reset). Thus, when the output of the temperature and humidity sensor 58 is +25° C. or less, the electromagnetic valve 47V opens the bypass pipe 47. In consequence, the refrigerant having the pressure thereof reduced by the expansion valve 34 does not flow through the second heat exchanger 33, all passes through the bypass pipe 47, and flows into the third heat exchanger 35.

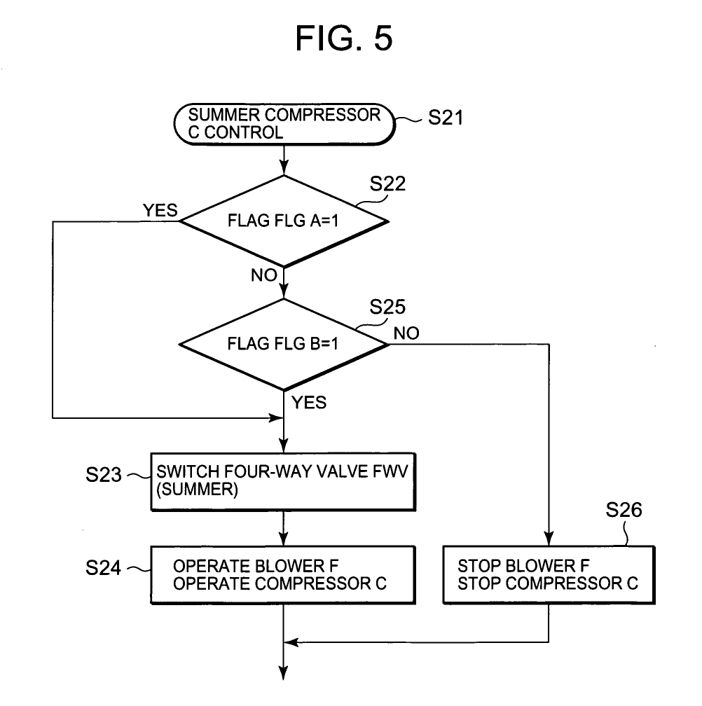

Next, control of the freezing unit 30 in summer will be described with reference to FIG. 5. In summer, the operation of the freezing unit 30 is controlled by operating the supply pump 62 or opening or closing the electromagnetic valve 47V. Specifically, in the above control (the control shown in FIGS. 3 and 4), the control is performed so that the operation is performed in a case where at least one of the flags A and B is set to 1 and so that the operation is sopped in a case where both the flags A and B are set to 0 (reset).

That is, when the power supply of the air cleaning apparatus 1 is turned on and the control of the freezing unit 30 is started in step S21 of FIG. 5, it is judged in step S22 of FIG. 5 whether or not the flag A is set to 1. Then, when the flag A is 1, in step S23 of FIG. 5, the four-way valve 38 (a four-way valve FWV shown in FIG. 5) is controlled (the four-way valve FWV is switched as shown in FIG. 5) so that the refrigerant compressed by the compressor 31 flows into the first heat exchanger 32 and so that the refrigerant from the third heat exchanger 35 is sucked into the compressor 31 as described above. Afterward, in step S24 of FIG. 5, the compressor 31 (a compressor C shown in FIG. 5) of the freezing unit 30 and the fan 32F (a blower F shown in FIG. 5) of the first heat exchanger 32 are operated. In consequence, as described above, the refrigerant flows through the freezing unit 30. It is to be noted that the refrigerant operates as described above, and hence description thereof is omitted here.

On the other hand, in a case where it is judged in the step S22 of FIG. 5 that the flag A is reset (the flag A is 0), the step shifts to step S25 of FIG. 5 to judge whether or not the flag B is set to 1. Then, in a case where the flag B is 1, the four-way valve 38 is controlled in the step S23 of FIG. 5, and the compressor 31 of the freezing unit 30 and the fan 32F are operated in the step S24 of FIG. 5 in the same manner as described above.

On the other hand, in a case where it is judged in the step S25 of FIG. 5 that the flag B is reset (the flag B is 0), the operations of the compressor 31 of the freezing unit 30 and the fan 32F of the first heat exchanger 32 are stopped in step S26 of FIG. 5. In consequence, the operation of the whole freezing unit 30 is stopped.

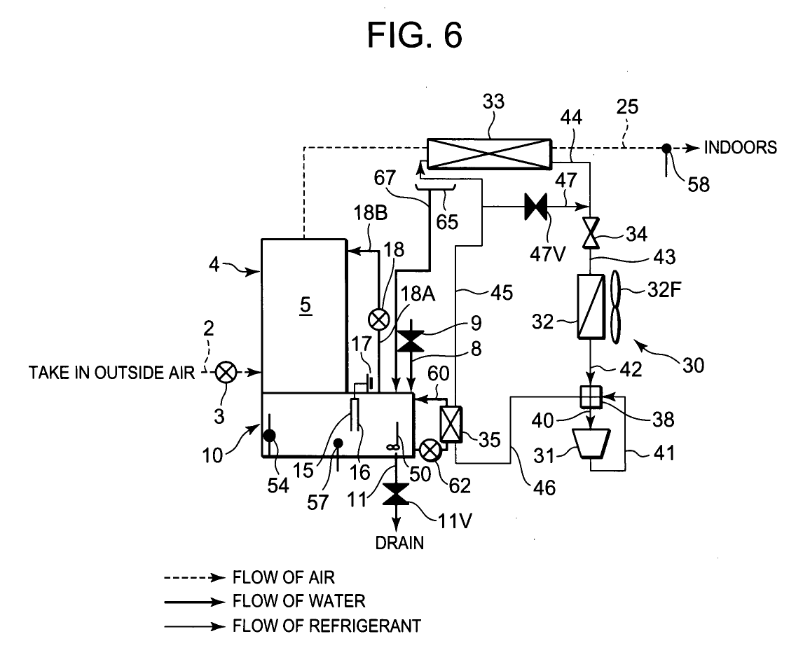

Next, there will be described an operation of the freezing unit 30 in a case where the air cleaning apparatus 1 of the present embodiment is used in winter when an outside air temperature is in a range of −30° C. to 10° C. (or in a district such as a cold district) with reference to FIG. 6. In this case, as shown in FIG. 6, the four-way valve 38 is controlled so that the refrigerant compressed by the compressor 31 flows into the third heat exchanger 35, and the refrigerant from the first heat exchanger 32 is sucked into the compressor 31. In consequence, the first heat exchanger 32 functions as an evaporator, and the third heat exchanger 35 and/or the second heat exchanger 33 function as a radiator. It is to be noted that in FIG. 6, broken-line arrows show a flow of the air to be treated, solid-line arrows show a flow of the refrigerant flowing through the freezing unit 30 during the operation in winter, and bold-line arrows show a flow of water, respectively.

That is, the refrigerant compressed by the operation of the compressor 31 and having a high temperature and high pressure is discharged from the compressor 31 via the refrigerant discharge pipe 41, and flows into the third heat exchanger 35 through the four-way valve 38. When the supply pump 62 is operated in the third heat exchanger 35 and the cleaning solution in the water tank 10 is supplied to the third heat exchanger 35, the refrigerant radiates heat to the cleaning solution supplied by the supply pump 62 in the third heat exchanger 35. In consequence, the cleaning solution radiates the heat. On the other hand, when the supply pump 62 is stopped, heat exchange between the refrigerant and the cleaning solution is hardly performed, and the refrigerant exits from the third heat exchanger 35 to flow into the refrigerant pipe 45.

In a case where the electromagnetic valve 47V of the bypass pipe 47 is closed, the refrigerant which has flowed into the refrigerant pipe 45 reaches the second heat exchanger 33 installed in the air supply duct 25. Then, in the second heat exchanger 33, the heat exchange between the refrigerant and the air to be treated flowing through the air supply duct 25 is performed to radiate the heat, and then the refrigerant flows into the refrigerant pipe 44.

On the other hand, when the electromagnetic valve 47V is opened to open the bypass pipe 47, the refrigerant from the third heat exchanger 35 does not flow through the second heat exchanger 33, and flows into the refrigerant pipe 44 through the bypass pipe 47.

Afterward, the refrigerant having the pressure thereof reduced by the expansion valve 34 then enters the first heat exchanger 32 to absorb there the heat from the surrounding air blown by the fan 32F, evaporates, and is then sucked from the refrigerant introduction pipe 40 into the compressor 31 via the refrigerant pipe 42 and the four-way valve 38. This cycle is repeated.

Here, during the operation in winter, the supply pump 62 is operated, and the electromagnetic valve 47V of the bypass pipe 47 is opened or closed, whereby the temperature of the cleaning solution is controlled into +5° C. or more to +15° C. or less. That is, the operation of the supply pump 62 is controlled based on the temperature of the cleaning solution in the water tank 10 detected by the temperature sensor 57, and the opening/closing of the electromagnetic valve 47V of the bypass pipe 47 is controlled based on the temperature and humidity of the air to be treated detected by the temperature and humidity sensor 58. A specific control operation in winter will hereinafter be described in detail.

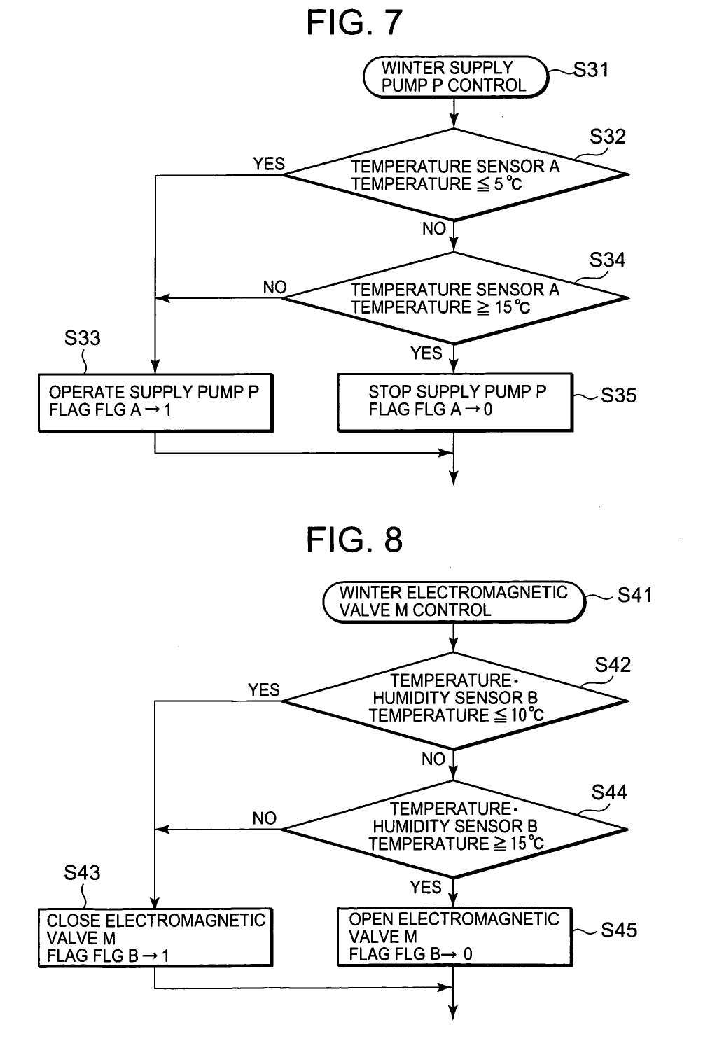

First, control of the supply pump 62 will be described with reference to FIG. 7. When the power supply of the air cleaning apparatus 1 is turned on and the control of the supply pump 62 (a supply pump P shown in FIG. 7) is started in step S31 of FIG. 7, it is judged in step S32 of FIG. 7 whether or not the temperature of the cleaning solution in the water tank 10 detected by the temperature sensor 57 (a temperature sensor A shown in FIG. 7) is +5° C. or less. Then, when the temperature of the cleaning solution detected by the temperature sensor 57 is +5° C. or less, the operation of the supply pump 62 is started, and a flag FLGA (hereinafter referred to as a flag A) is set to 1 in step S33 of FIG. 7.

Thus, when the temperature of the cleaning solution in the water tank 10 detected by the temperature sensor 57 is +5° C. or less, the supply pump 62 is operated. In consequence, the cleaning solution in the water tank 10 is supplied to the third heat exchanger 35, and the heat exchange between the refrigerant and the cleaning solution is performed in the third heat exchanger 35. In consequence, the refrigerant flowing through the third heat exchanger 35 radiates the heat to heat the cleaning solution, so that freezing of the cleaning solution in the water tank 10 can be prevented in advance.

On the other hand, in a case where it is judged in the step S32 of FIG. 7 that the temperature of the cleaning solution detected by the temperature sensor 57 is higher than +5° C., it is judged in step S34 of FIG. 7 whether or not the output of the temperature sensor 57 is +15° C. or more. Here, when the output of the temperature sensor 57 is lower than +15° C., in the step S33 of FIG. 7, the supply pump 62 is operated, and the flag A is set to 1 as described above. On the other hand, in a case where it is judged in the step S34 of FIG. 7 that the temperature is +15° C. or more, in step S35 of FIG. 7, the operation of the supply pump 62 is stopped, and the flag A is set to 0 (i.e., the flag A is reset).

Thus, in a case where the temperature of the cleaning solution in the water tank 10 detected by the temperature sensor 57 rises to +15° C. or more, the supply pump 62 is stopped, so that the heat exchange between the refrigerant and the cleaning solution is not performed in the third heat exchanger 35. In consequence, a disadvantage that the cleaning solution in the water tank 10 is heated more than necessary can be avoided.

Next, the control of the electromagnetic valve 47V in winter will be described with reference to FIG. 8. When the power supply of the air cleaning apparatus 1 is turned on and the control of the electromagnetic valve 47V (an electromagnetic valve M shown in FIG. 8) is started in step S41 of FIG. 8, it is judged in step S42 of FIG. 8 whether or not the temperature of the air to be treated in the air supply duct 25 detected by the temperature and humidity sensor 58 (a temperature and humidity sensor B shown in FIG. 8) is +10° C. or less. Then, in a case where the temperature of the air to be treated detected by the temperature and humidity sensor 58 is +10° C. or less, in step S43 of FIG. 8, the electromagnetic valve 47V is closed, and a flag FLGB (hereinafter referred to as the flag B) is set to 1.

Thus, in a case where the temperature of the air to be treated detected by the temperature and humidity sensor 58 is +10° C. or less, the bypass pipe 47 is closed by the electromagnetic valve 47V. In consequence, the refrigerant from the third heat exchanger 35 does not flow through the bypass pipe 47, and all of the refrigerant flows into the second heat exchanger 33 installed in the air supply duct 25 to radiate the heat to the air to be treated flowing around the second heat exchanger 33, and further radiates the heat. In consequence, the air to be treated can be heated. Therefore, the air to be treated supplied indoors from the air supply duct 25 can be heated to perform or assist indoor warming.

On the other hand, in a case where it is judged in the step S42 of FIG. 8 that the temperature of the air to be treated in the air supply duct 25 detected by the temperature and humidity sensor 58 is higher than +10° C., it is judged in step S44 of FIG. 8 whether or not the output of the temperature and humidity sensor 58 is +15° C. or more. Then, when the output of the temperature and humidity sensor 58 is lower than +15° C., in the step S43 of FIG. 8, the electromagnetic valve 47V is closed and the flag B is set to 1 as described above.

On the other hand, in a case where it is judged in the step S44 of FIG. 8 that the output of the temperature and humidity sensor 58 is +15° C. or more, in step S45 of FIG. 8, the electromagnetic valve 47V is opened, and the flag B is set to 0 (i.e., the flag B is reset). Thus, when the output of the temperature and humidity sensor 58 is +15° C. or more, the electromagnetic valve 47V opens the bypass pipe 47. In consequence, the refrigerant from the third heat exchanger 35 does not flow through the second heat exchanger 33, all passes through the bypass pipe 47, and flows into the refrigerant pipe 44 to reach the expansion valve 34.

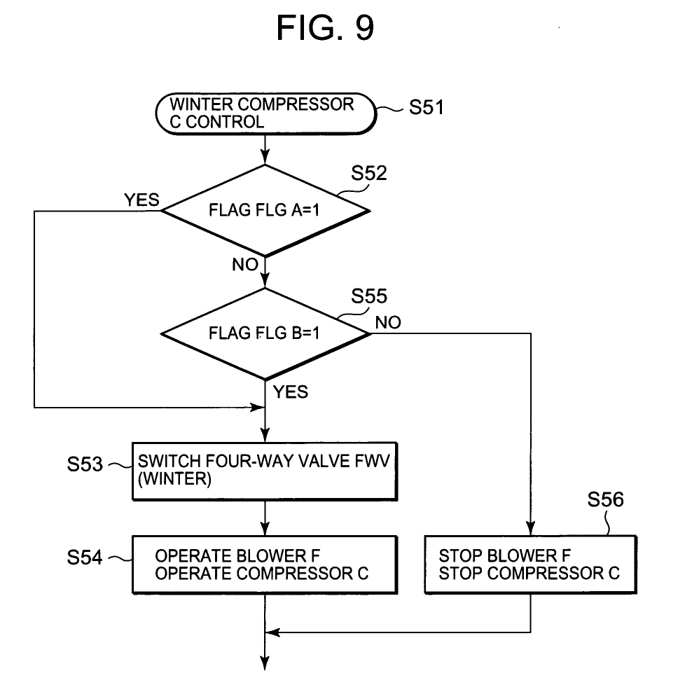

Next, the control of the freezing unit 30 in winter will be described with reference to FIG. 9. In winter, the operation of the freezing unit 30 is controlled by operating the supply pump 62 or opening or closing the electromagnetic valve 47V. Specifically, in the above control (the control shown in FIGS. 7 and 8), the control is performed so that the operation is performed in a case where at least one of the flags A and B is set to 1 and so that the operation is sopped in a case where both the flags A and B are set to 0 (i.e., both the flags A and B are reset).

That is, when the power supply of the air cleaning apparatus 1 is turned on and the control of the freezing unit 30 is started in step S51 of FIG. 9, it is judged in step S52 of FIG. 9 whether or not the flag A is set to 1. Then, when the flag A is 1, in step S53 of FIG. 9, the four-way valve 38 (a four-way valve FWV shown in FIG. 9) is controlled (the four-way valve FWV is switched as shown in FIG. 9) so that the refrigerant compressed by the compressor 31 flows into the third heat exchanger 35 and so that the refrigerant from the first heat exchanger 32 is sucked into the compressor 31 as described above. Afterward, in step S54 of FIG. 9, the compressor 31 (a compressor C shown in FIG. 9) of the freezing unit 30 and the fan 32F (a blower F shown in FIG. 9) of the first heat exchanger 32 are operated. In consequence, as described above, the refrigerant flows through the freezing unit 30. It is to be noted that the refrigerant operates as described above, and hence description thereof is omitted here.

On the other hand, in a case where it is judged in the step S52 of FIG. 9 that the flag A is reset (the flag A is 0), the step shifts to step S55 of FIG. 9 to judge whether or not the flag B is set to 1. Then, in a case where the flag B is 1, the four-way valve 38 is controlled in the step S53 of FIG. 9, and the compressor 31 of the freezing unit 30 and the fan 32F are operated in the step S54 of FIG. 5 in the same manner as described above.

On the other hand, in a case where it is judged in the step S55 of FIG. 9 that the flag B is reset (the flag B is 0), the operations of the compressor 31 of the freezing unit 30 and the fan 32F of the first heat exchanger 32 are stopped in step S56 of FIG. 9. In consequence, the operation of the whole freezing unit 30 is stopped.

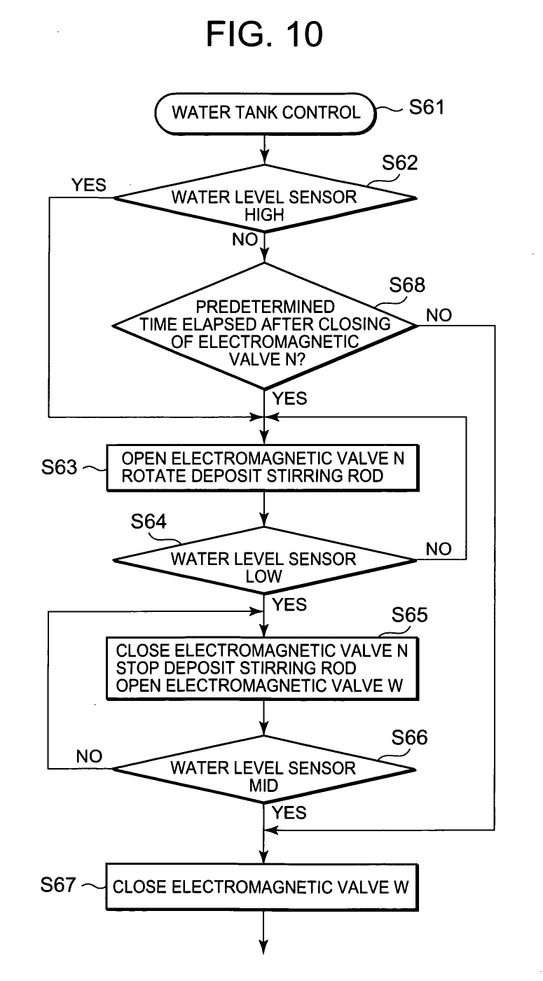

Next, discharge of the sediments deposited in the water tank 10 and control of water supply into the water tank 10 will be described. In the water tank 10, fine matters such as soil and sandblast collected by the contact between the cleaning solution and the air to be treated in the cleaning tower 4 are deposited as described above, so that these matters need to be periodically discharged, and water needs to be supplied to the water tank 10. To solve the problem, in the present embodiment, the water supply into the water tank 10 of the air cleaning apparatus 1 and the discharge of the sediments from the water tank 10 are controlled. A control operation will be described with reference to FIG. 10.

First, in a case where the power supply of the air cleaning apparatus 1 is turned on and the control of the water tank 10 is started in step S61 of FIG. 10, it is judged in step S62 of FIG. 10 whether or not a water level in the water tank 10 detected by the water level sensor 54 is a predetermined high level (HIGH shown in FIG. 10). Then, in a case where the water level of the cleaning solution in the water tank 10 detected by the water level sensor 54 is the predetermined high level, in step S63 of FIG. 10, the electromagnetic valve 11V (an electromagnetic valve N shown in FIG. 10) of the drain port 11 is opened, and the deposit stirring rod 52 is operated (rotated). In consequence, the drain port 11 is opened to discharge, from the drain port 11, the sediments accumulated around the drain port 11 together with the cleaning solution in the water tank 10. Here, as in the present embodiment, the whole bottom part of the water tank 10 is configured so as to tilt downward to this drain port 11, and the sediments are stirred with the deposit stirring rod 52, whereby the discharge of the sediments from the drain port 11 can be promoted.

In a case where the drain port 11 is opened and the deposit stirring rod 52 is operated (rotated) in the step S63 of FIG. 10, it is then judged in step S64 whether or not the water level of the cleaning solution in the water tank 10 detected by the water level sensor 54 lowers to a predetermined low level (LOW shown in FIG. 10). Then, when the water level of the cleaning solution in the water tank 10 detected by the water level sensor 54 lowers to the predetermined low level, in step S65 of FIG. 10, the electromagnetic valve 11V of the drain port 11 is closed, the rotating operation of the deposit stirring rod 52 is stopped, and the water supply valve 9 (an electromagnetic valve W shown in FIG. 10) is opened. This water supply valve 9 is opened to open the water supply passage 8, whereby the water is supplied from a water source into the water tank 10. Simultaneously with the closing of the electromagnetic valve 11V, a time starts to be counted.

Then, in a case where it is judged in step S66 that the water level detected by the water level sensor 54 is a predetermined middle level (MID shown in FIG. 10) set between the low level and the high level, in step S67 of FIG. 10, the water supply valve 9 is closed to stop the water supply from the water supply passage 8.

On the other hand, in a case where it is judged in the step S62 of FIG. 10 that the water level of the cleaning solution in the water tank 10 detected by the water level sensor 54 is lower than the predetermined high level, it is judged in step S68 of FIG. 10 whether or not a predetermined time has elapsed after the electromagnetic valve 11V was closed. Then, in a case where the predetermined time has been counted after the electromagnetic valve 11V was closed, the step shifts to the step S63 to repeat the above control (the electromagnetic valve 11V is opened, the deposit stirring rod 52 is operated (rotated), and then the control shifts to the step S64). In consequence, regardless of the water level in the water tank 10, the electromagnetic valve 11V is periodically opened to open the drain port 11, so that the sediments in the water tank 10 can be discharged.

Moreover, in a case where it is judged in the step S68 that the predetermined time has not elapsed after the electromagnetic valve 11V was closed, the step shifts to the step S67 of FIG. 10 to close the water supply valve 9, whereby the water supply from the water supply passage 8 is stopped.

Furthermore, in a case where it is judged in the step S64 that the water level in the water tank 10 detected by the water level sensor 54 does not lower to the predetermined low level, the step returns to the step S63, and the control of the steps S63, 64 is repeated until the water level in the water tank 10 lowers to the predetermined low level.

Furthermore, in a case where it is judged in the step S66 that the water level detected by the water level sensor 54 is not the predetermined middle level (MID shown in FIG. 10), the step returns to the step S65, and the control of the steps S65, 66 is repeated until the water level in the water tank 10 becomes the predetermined middle level. It is to be noted that the above control, that is, the control shown in FIGS. 3 to 5 and 10 in summer or the control shown in FIGS. 7 to 10 in winter is performed continuously or in parallel during the operation of the air cleaning apparatus 1.

An evaluation test to treat an ammonia gas (odor) having a concentration of 500 ppm for 90 minutes by use of the air cleaning apparatus 1 described above in detail was performed to verify a treatment effect of the apparatus. In this case, the cleaning solution was jetted from the shower head 20 into the cleaning tower 4 having a diameter of 280 mm and a height of 1 m by the supply pump 18 at a rate of 2.5 L/min, and the ammonia gas was supplied into the cleaning tower 4 at a rate of 10 L/min. Then, control was performed so that a constant current of 1 A (a current density of 23.8 mA/cm²) flowed through the electrodes 15, 16 from the power source 17. In this case, as the water in the water tank 10, water to which 1.0% of sodium chloride was added was used. It is to be noted that 10 L of water was used in the whole air cleaning apparatus 1.

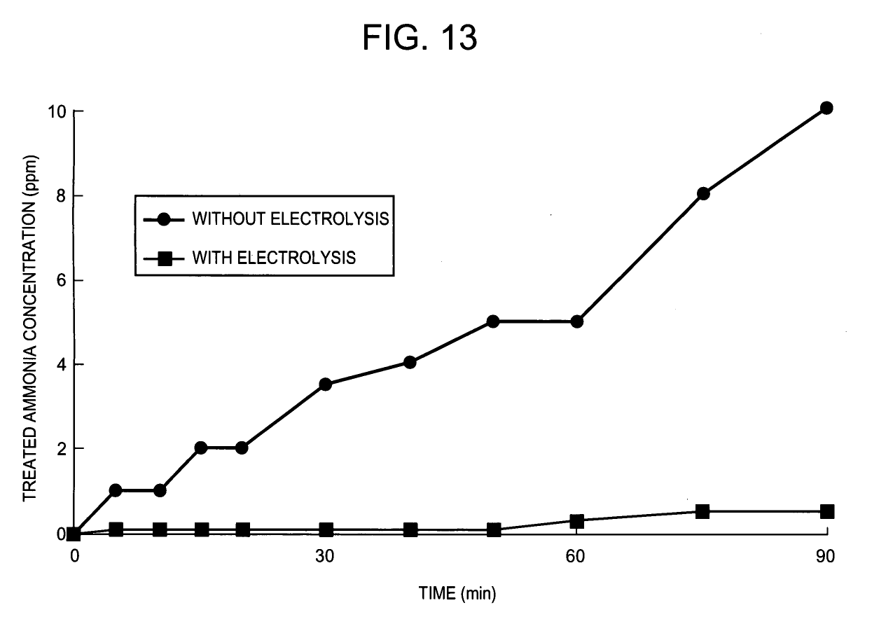

Black quadrangular points of FIG. 13 show a change of an ammonia gas concentration with time in a case where the ammonia gas was brought into contact with the cleaning solution including the active oxygen species obtained by use of the air cleaning apparatus 1 described above in detail, that is, the electrolytic treatment. Moreover, black circle points of FIG. 13 show a change of the ammonia gas concentration with time in a case where a commercially available aqueous active oxygen species solution was used as the cleaning solution instead of obtaining the cleaning solution including the active oxygen species by the electrolytic treatment.

As shown in FIG. 13, even when the commercially available aqueous active oxygen species solution or the like is used as the cleaning solution without performing any electrolytic treatment and this solution is brought into contact with the ammonia gas, the ammonia gas can be reduced to several ppm. However, a high removal ratio cannot be maintained for a long time. It has been found that the electrolytic treatment is performed as in the present embodiment, whereby 99% or more of the ammonia gas can be removed, and the effect can be continued for a long time.

According to the present invention, as described above in detail, regardless of seasons, weather, environmental conditions and the like, toxic substances such as odor, pollen, allergen, a VOC, a pesticide and oxidant, fine matters such as soil and sandblast and the like in the air to be treated can efficiently be removed using the air cleaning apparatus 1 throughout the year in any district of the world.

It is to be noted that in the present embodiment, the cleaning solution including the active oxygen species is formed by the electrolysis of the tap water in the water tank 10, but a method for forming the cleaning solution including the active oxygen species by the electrolysis as described in the embodiment is merely one example, and the invention according to the first to sixth aspects or the ninth aspect is not necessarily limited to this example. For example, even when the cleaning solution including the active oxygen species is formed by a photocatalyst or gas-phase electric discharge, the invention of the first to sixth aspects or the ninth aspect is effective.

What is claimed is:

1. An air cleaning apparatus which brings air to be treated into contact with a cleaning solution including active oxygen species to purify the air to be treated, comprising:
a water tank which stores the cleaning solution; and
a temperature controller which controls a temperature of the cleaning solution stored in the water tank.

2. The air cleaning apparatus according to claim 1, wherein the temperature controller includes cooling/heating means for cooling or heating the cleaning solution stored in the water tank, and controls the temperature of the cleaning solution into 0° C. or more to 40° C. or less.

3. The air cleaning apparatus according to claim 2, wherein the temperature controller controls the temperature of the cleaning solution into 5° C. or more to 15° C. or less.

4. The air cleaning apparatus according to claim 2, wherein the temperature controller controls the temperature of the cleaning solution into 20° C. or more to 25° C. or less.

5. The air cleaning apparatus according to claim 1 to 4, wherein the temperature controller includes dehumidifying means for dehumidifying the air to be treated brought into contact with the cleaning solution and then supplied to an air supply space.

6. The air cleaning apparatus according to claim 5, which further comprises means for collecting, in the water tank, water condensed and formed by the dehumidifying means.

7. The air cleaning apparatus according to claim 1 to 6, wherein the cleaning solution is obtained by electrolyzing the water in the water tank.

8. The air cleaning apparatus according to claim 7, wherein the water tank includes a depositing section which collects the cleaning solution brought into contact with the air to be treated, and an electrolysis section connected to the depositing section and provided with electrodes which electrolyze the water in the water tank, and the depositing section has a drain port opened/closed by a valve, and tilts downward to the drain port.

9. The air cleaning apparatus according to any one of claims 1 to 8, wherein each of the active oxygen species is one selected from the group consisting of hypochlorous acid, ozone, hydroxyl radicals and combinations thereof.
